(19) United States
(12) Patent Application Publication
(10) Pub. No.: US 2002/0198529 A1
(43) Pub. Date: Dec. 26, 2002

(54) Optimizing patellar femoral mechanics through alternative depth referencing

(76) Inventor: Michael A. Masini, Ann Arbor, MI (US)

Correspondence Address: John G Posa, Gifford Krass Groh Sprinkle Patmore, Anderson & Citkowski PC, 280 N Old Woodward Avenue Suite 400, Birmingham, MI, 48009, US

(21) Appl. No.: 09/523,503

(22) Filed: Mar. 10, 2000

Related U.S. Application Data
(60) Provisional application No. 60/120,062, filed on Feb. 16, 1999.
(60) Provisional application No. 60/124,062, filed on Mar. 12, 1999.

(51) Int. Cl. A61B 17/00

(52) U.S. Cl. 606/87

(57) ABSTRACT

In knee-replacement surgery, restoration is achieved with respect to the patellar femoral joint in the distal plane is, thereby optimizing patellar femoral mechanics. The depth of the trochlea is increased with increasing implant size. In the preferred embodiment, this is achieved by referencing the extent of the lateral femoral condyle or trochlear region, and resecting the distal femur in accordance with the extent of the lateral femoral condyle or trochlear region. As an alternative, the invention provides for distal femoral and proximal tibial components having bone-contacting and articulating surfaces which account for the measured extent of the lateral femoral condyle or trochlear region. A method of preparing a distal femur according to the invention includes the steps of installing a rod or stem within the intramedullary canal, and attaching a referencing fixture thereto. The extent of the lateral femoral condyle or trochlear region is measured using the referencing fixture, and the distal femur is resected in accordance with the extent of the lateral femoral condyle or trochlear region. The method typically further includes the step of placing a spacer between the referencing fixture and the lateral femoral condyle or trochlear region. The preferred alternative embodiment of the invention involving the use of modified components proceeds similarly, except that after measuring the extent of the lateral femoral condyle or trochlear region using the referencing fixture, distal femoral and proximal tibial components are implanted having bone-contacting and articulating surfaces which take the measurement into account.

### REFERENCE TO RELATED APPLICATION

This application claims priority of U.S. provisional application Serial No. 60/120,062, filed Mar. 12, 1999, the entire contents of which is incorporated herein by reference.

### FIELD OF THE INVENTION

This invention relates generally to orthopedic surgery and, in particular, to alternative depth referencing in conjunction with knee-replacement surgery.

### BACKGROUND OF THE INVENTION

Whether for primary or revision arthroplasty, cutting guides are typically employed to ensure that the bone saw performs resections corresponding to mating surfaces of the prosthetic component. For example, in a femoral knee replacement, cutting guides or blocks are temporarily secured to the distal end of the femoral shaft, and include slots into which the blade of an oscillating saw is inserted to shape the end of the bone in accordance with corresponding surfaces of the prosthetic element.

The distal end portion of a natural femur terminates in two bulbous protrusions termed the medial and lateral condyles, which mate and engage with corresponding surfaces in the proximal end of the tibia. As a result of disease or injury, these mating surfaces, ordinarily smooth and cushioned by an intervening cartilage layer, disintegrate and/or become misshapen, resulting in restricted movement and pain.

To ameliorate these conditions, the orthopedic surgeon removes the unhealthy bone stock and replaces it with one or more metallic components which adhere to appropriately prepared bone surfaces and approximate the outer, cortical layer of a healthy bone. To prepare the existing damaged or diseased bone to accept the implant components, various resections are made in a predetermined manner in correspondence with the inner surfaces of the implant.

Using the example of a distal femur, a saw guide is used to form resected surfaces typically including a distal cut, anterior and posterior cuts, and perhaps anterior or posterior chamfer cuts. Although these cuts represent resections made in conjunction with a standard implant technique, more, fewer or different surfaces may be required, depending upon the level of deterioration or other circumstances.

Depending upon the saw guide used, either the cuts associated with only one of the condyles may be resected, or, alternatively, a guide having a dual set of slots may be utilized to trim both condyles simultaneously. A singular type fixture is shown, for example, in U.S. Pat. No. 5,122,144, whereas guides having double sets of slots are shown in U.S. Pat. Nos. 5,129,909 and 5,364,401. Numerous other examples are evident in the prior art, some of which are in commercial usage.

Certain problems may arise in making the aforementioned resections, particularly with respect to placement of the distal cut in conjunction with total knee arthroplasty. A distal femur usually exhibits about seven degrees of valgus for a man, and about nine degrees of valgus for a woman. The corresponding tibia usually requires three degrees of varus. As a consequence, the total alignment for a man is about four degrees of valgus, whereas the alignment for a woman is about six degrees of valgus.

One problem arises from the fact that placement of the distal cutting guide is usually based on the most prominent condyle, which tends to be the medial condyle. Since the proximal tibia is typically removed as part of a joint replacement, more bone is ordinarily removed laterally as compared to medially, so that the resulting configuration is no longer varus, but neutral, or zero degrees. This means that relative to the femur, more bone must be removed medially than laterally. This situation has implications to the flexion and extension gaps relative to the patella femoral joint.

Reference is made to FIG. 1, which is an anterior-posterior view of a distal femur, depicted generally at 100, with the medial femoral condyle being shown at 102. The instrumentation associated with current procedures includes an intramedullary rod 103 on which there is mounted distal plate 104. Once plate 104 contacts the distal femur, it is set into place. Then a distal cutting guide 202 having one or more slots is positioned relative to the plate on a plate extension 203 which typically includes markings enabling the surgeon to determine how much bone will be removed. The distal cutting guide slides in a distal-to-proximal direction on 203 until a desired depth is selected. FIG. 2 is a lateral view of the same device 203 viewed from an oblique perspective showing the extension arm on which the cutting guide 202 can slide proximal to distal. Item 201 is the intramedullary rod.

In any case, since the plate rests against the most proud condyle, the chosen level will lead to more resection from the medial side and less from the lateral side. Depending upon patient anatomy, additional bone may or may not be removed from the region of the trochlea, which is the central depression between the two distal portions.

FIG. 3A shows the expected result without instrumentation in the AP plane, that is, how more of the proximal tibia will be trimmed laterally than medially, and how, correspondingly, more of the distal femur will be resected medially than laterally, so as to create a symmetric extension gap. FIG. 3B is a drawing which helps to understand the problems caused by the goal of a symmetric extension gap. A distal advancement of the lateral condyle is evident, which is shown between the two arrows. This also results in a distal advancement of the trochlea, depending on the size of the patient.

FIG. 4 represents the effect this approach has on the extensor mechanism with the patella intact. In essence, the extensor mechanism is displaced more distally with respect to the patella. The increased moment arm of the extensor mechanism results in increased force on the patella in flexion, increased potential for wear, loss of flexion of the joint, and altered tracking of the patella (i.e., patella tilt).

### SUMMARY OF THE INVENTION

In knee-replacement surgery, the present invention allows for the creation of a symmetric extension gap while providing restoration of the joint line with respect to patellar femoral joint in the distal plane, thereby optimizing patellar femoral mechanics. Broadly, in meeting this objective, the depth of the trochlea is increased with increasing implant size. In the preferred embodiment, this is achieved by referencing the extent of the lateral femoral condyle or trochlear region, and resecting the distal femur in accordance with the extent of the lateral femoral condyle or trochlear region. As an alternative, the invention provides for distal femoral and proximal tibial components having bone-contacting and articulating surfaces which account for the measured extent of the lateral femoral condyle or trochlear region.

A method of preparing a distal femur according to the invention includes the steps of installing a rod or stem within the intramedullary canal, and attaching a referencing fixture thereto. The extent of the lateral femoral condyle or trochlear region is measured using the referencing fixture, and the distal femur is resected in accordance with the extent of the lateral femoral condyle or trochlear region. The method typically further includes the step of placing a spacer between the referencing fixture and the lateral femoral condyle or trochlear region. The preferred alternative embodiment of the invention involving the use of modified components proceeds similarly, except that after measuring the extent of the lateral femoral condyle or trochlear region using the referencing fixture, distal femoral and proximal tibial components are implanted having bone-contacting and articulating surfaces which take the measurement into account.

### BRIEF DESCRIPTION OF THE DRAWINGS

FIG. 1 is an anterior-posterior view of a distal femur including an intramedullary rod on which there is mounted a prior-art distal plate used for depth referencing;

FIG. 2 is a lateral view of the device of FIG. 1 viewed from an oblique perspective showing an extension arm on which a cutting guide can slide proximal to distal;

FIG. 3A shows how the proximal tibia is trimmed more laterally than medially according to current techniques;

FIG. 3B is a drawing which helps to understand the problems caused by the use of the medial condyle as the reference for the distal resection;

FIG. 4 represents the effect of a symmetric extension gap on the extensor mechanism with the patella intact;

FIG. 5 illustrates a distal cutting guide according to the invention which references the lateral femoral condyle as opposed to the medial condyle;

FIG. 6 is a drawing that illustrates an alternative embodiment wherein an appropriately shaped spacer is placed in the region of the trochlea so as to perform the distal resection relative to the trochlea;

FIG. 7 shows how a final implant may be modified as opposed altered resections according to the invention;

FIG. 8A shows how the thickness of a tibial implant may be made thicker while keeping the tibial insert symmetric;

FIG. 8B shows how the thickness of a tibial spacer may be made thicker while keeping the implant or tray largely symmetric;

FIG. 9A depicts the current situation involving symmetric medial and lateral condyles and the corresponding trochlea;

FIG. 9B shows how, as implant size gets larger, the distance between the distal portions of both condyles and the trochlea increases by virtue of the invention; and

FIG. 10 depicts an alternative embodiment of the invention includes a gauge moveable medially to laterally to reference either condyle or the trochlear region.

### DETAILED DESCRIPTION OF THE INVENTION

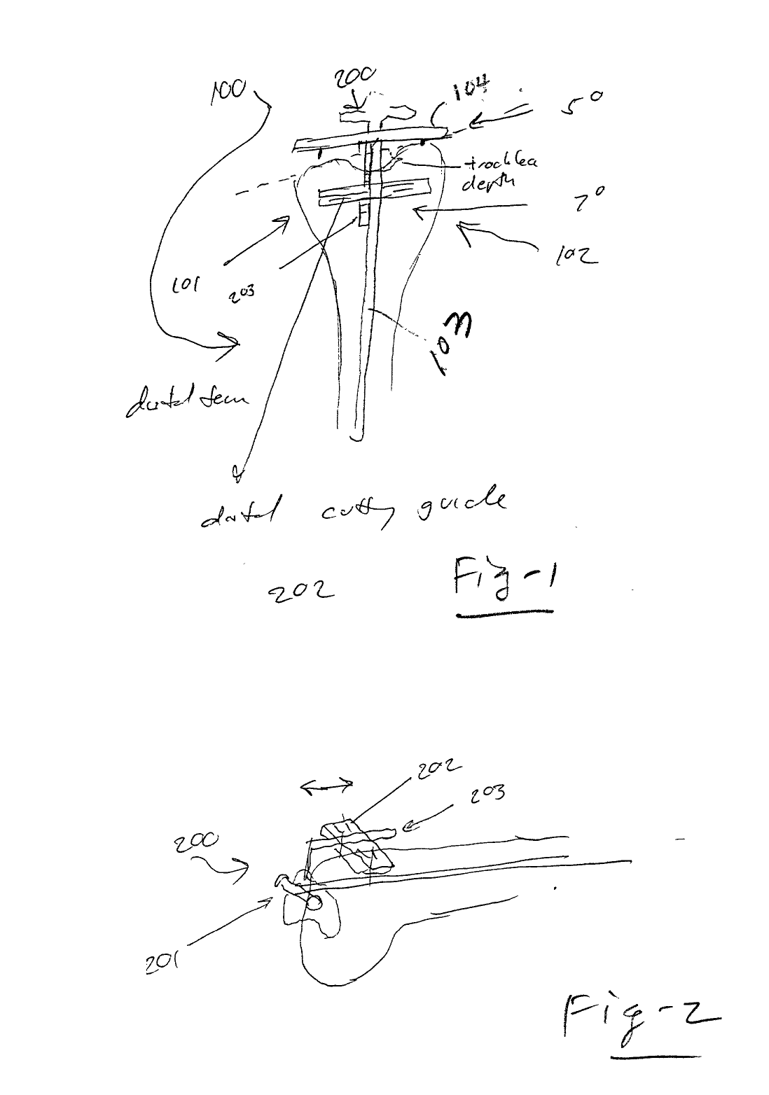

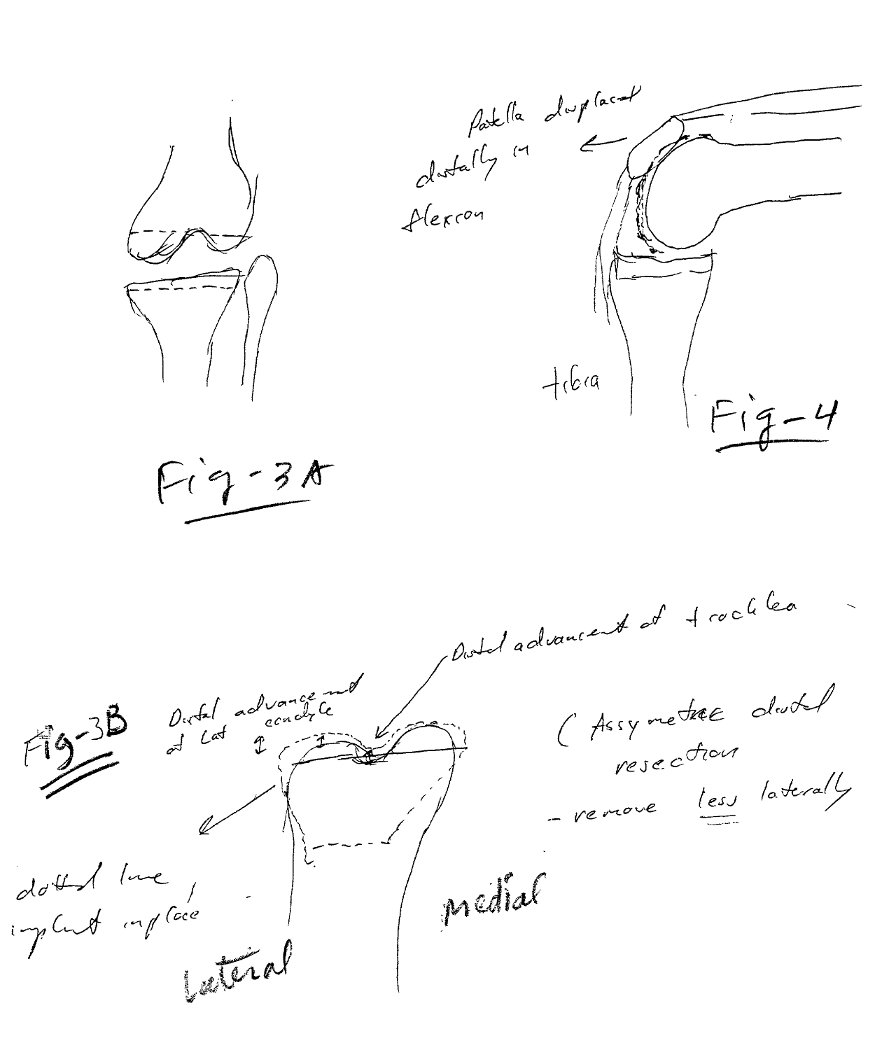

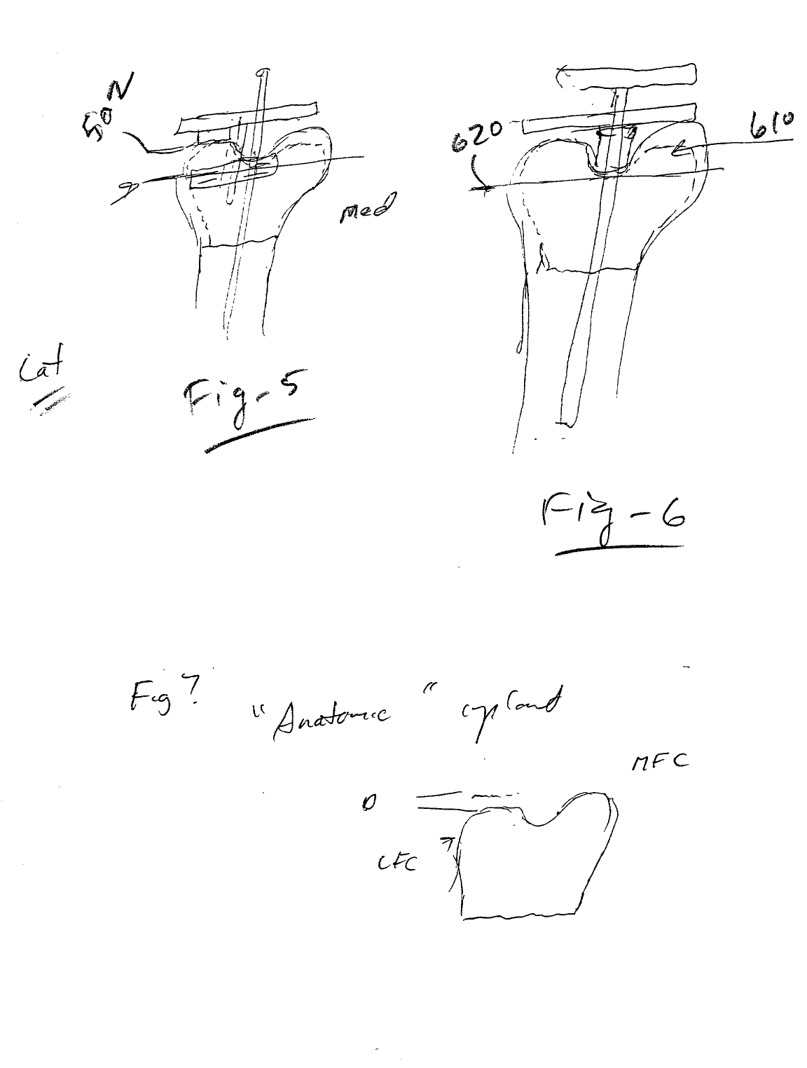

Having discussed the deficiencies of the prior art with reference to FIGS. 1 through 4, the reader's attention is now directed to FIG. 5, which illustrates a distal cutting guide according to the invention which references the lateral femoral condyle as opposed to the medial condyle. In the event that the lateral femoral condyle is normally formed, a spacer 502 may be positioned between the extent of the lateral condyle and the distal plate, as shown. The amount of bone resected then would correspond to the difference between this point and the position of the cutting guide. If some bone loss were to occur laterally, this could be compensated through the use of a thicker spacer.

As an alternative, a preferred embodiment is seen in FIG. 6, wherein an appropriately shaped spacer is placed in the region of the trochlea so as to perform the distal resection relative to the trochlea. In this manner, one would be sure that when one restored the ultimate final implant, that it was restored with respect to the patella femoral joint in the distal plane. The spacer 610 is used to reference the trochlea following osteophyte removal. Line 620 represents the level of resection to restore implant, bone construct to the level of the normal trochlea.

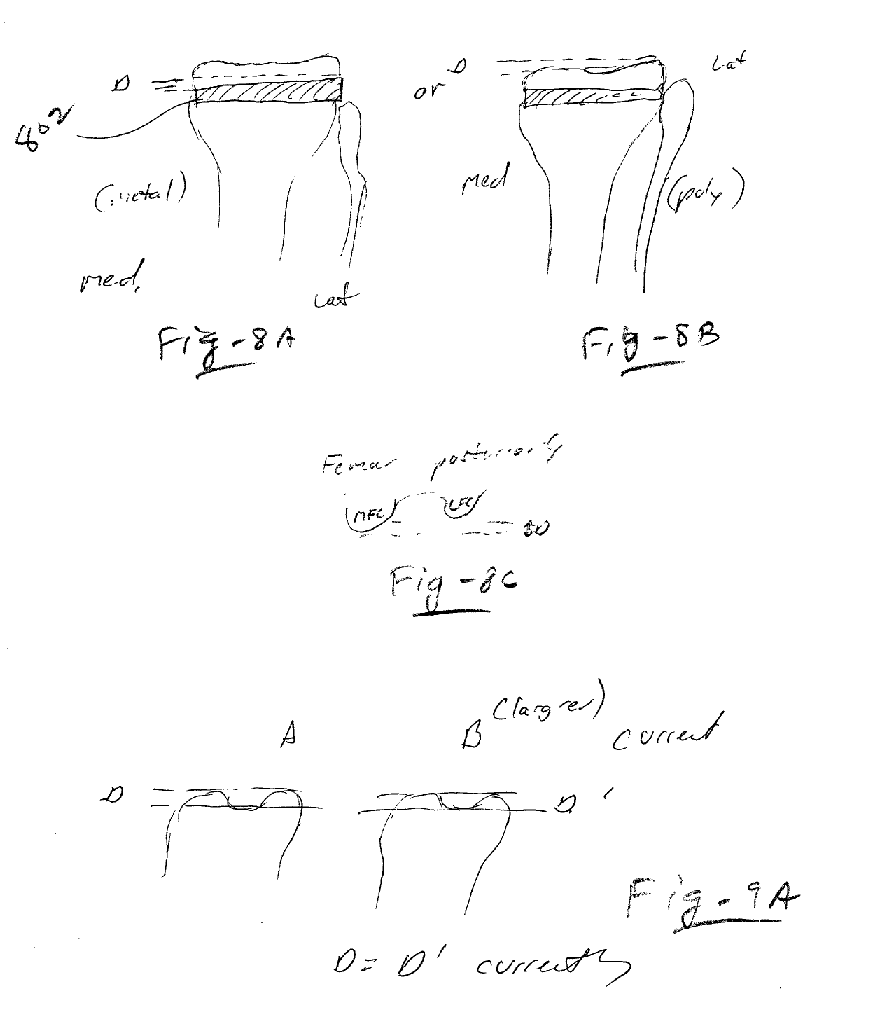

FIG. 7 shows how one could actually alter the final implant as opposed to necessarily altering the cut. In this case, a distal position of the lateral femoral condyle would be less than the medial femoral condyle by an amount D. In addition, the trochlea would be deeper as well. A slight resection, of 10 millimeters, could be performed to that thickness of metal medially. Less metal would be restored laterally, on the order of 8 millimeters, for example, and the trochlea then correspond as well.

Using this approach, one would also have to make alterations to the tibial surface. This could be accomplished in several ways. One could have the metal thicker, as seen in FIG. 8A, in which case the insert 802, typically polyethylene, would remain symmetric. Alternatively, the metal could be made symmetric, with the spacer also being made thicker by the distance D, as seen in FIG. 8B. This would correct for any incongruity with respect to the extension gap, while still allowing for appropriate mechanics of the patella femoral joint.

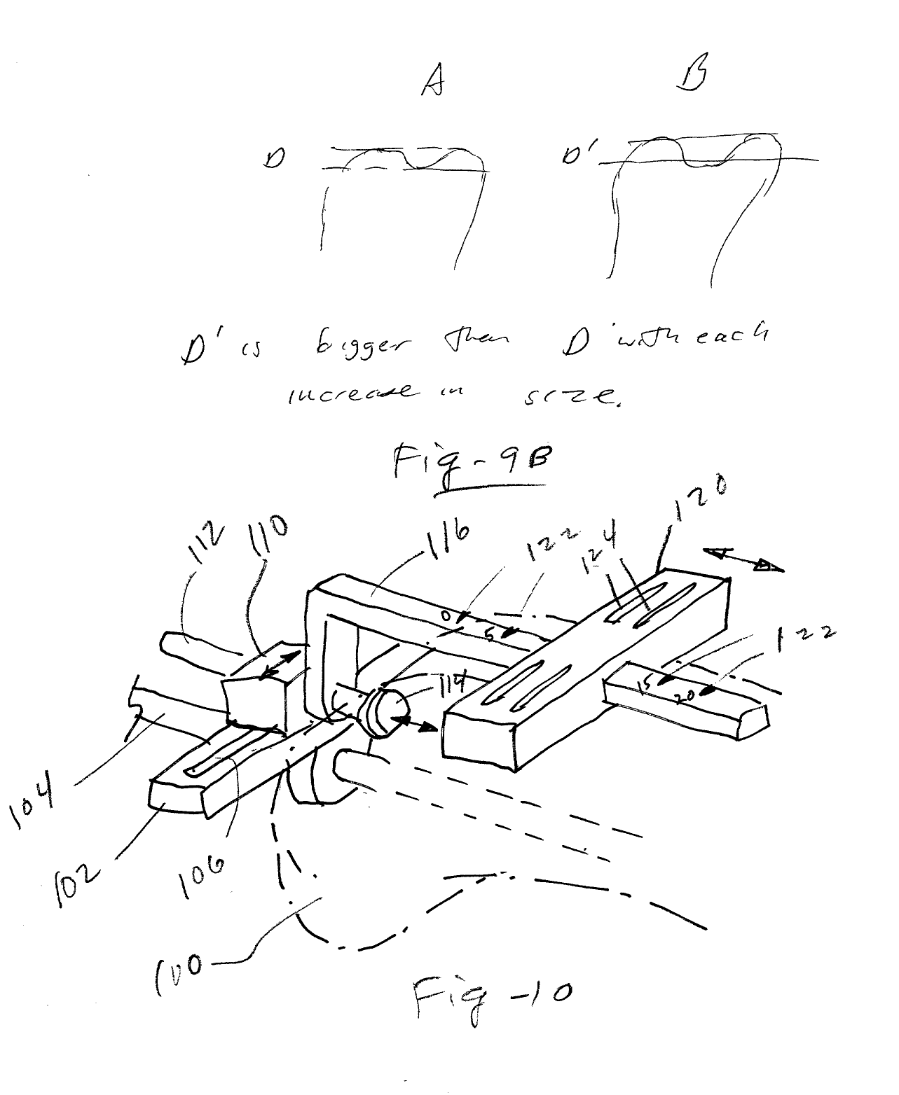

By way of review, FIG. 9A represents, once again, the current situation involving symmetric medial and lateral condyles and the corresponding trochlea. According to the invention, the trochlea depths, which are represented by D and D′ prime would change for a given size. As such, when the size gets larger, such as size B in the drawing of FIG. 9B, the distance between the distal portions of both condyles and the trochlea remains the same. However, according to the invention, as the size of implant increases, the depth of the trochlea increases correspondingly so as to optimize the patella femoral mechanics.

FIG. 10 illustrates, from an oblique perspective, an embodiment of the invention including a medial-lateral slide enabling referencing to take place between either condyle or the trochlear region. The device includes a fixture 102 that rides on an intermedullary rod 104 including a groove 106 which receives a medial-lateral slide 110. The slide 110 further includes a slidable member 112, adjustable longitudinally in a manner generally parallel to the rod 104, including a referencing surface 114 and an angled member 116, including a cutting guide 120, which moves on the member 116, the member 116 further including calibrations 122 indicative of cutting depth. Note that the angled member 116 is not slidingly attached to the rod 112, but is rigidly attached thereto, such that as the assembly including rod and reference surface 114 moves longitudinally with respect to the bone, the member 116 moves therewith. In operation, the assembly containing rod 112, surface 114, member 116 and cutting block 120 may be moved medial to lateral, enabling the surface 114 to reference either condyle or the trochlear region of the bone 100. Having selected the reference point, the block 120 may be moved along member 116, taking note of the markings 122 which will be indicative of cutting depth. Upon selecting a desired cutting depth, one or more of the slots 124 may be used to resect either or both of the condyles, as the case may be.

I claim:

1. A method of installing a prosthesis onto a distal femur having an intramedullary canal so that the joint is restored with respect to the patellar femoral joint in the distal plane, the method comprising the steps of:
installing a rod or stem having a first end positioned within the intramedullary canal and a second end that remains exposed;
coupling a referencing fixture to the second end of the rod or stem;
measuring the extent of the lateral femoral condyle or trochlear region using the referencing fixture; and
installing the prosthesis in accordance with the extent of the lateral femoral condyle or trochlear region, as measured with the referencing fixture.

2. The method of claim 1, wherein the step of installing the prosthesis includes the step of resecting the distal femur in accordance with the extent of the lateral femoral condyle or trochlear region, as measured with the referencing fixture.

3. The method of claim 2, including the step of placing a spacer between the referencing fixture and the lateral femoral condyle or trochlear region.

4. The method of claim 1, wherein the step of installing the prosthesis includes the step of providing distal femoral and proximal tibial components having bone-contacting and articulating surfaces which account for the extent of the lateral femoral condyle or trochlear region, as measured with the referencing fixture.

5. A method of preparing a distal femur having an intramedullary canal for primary arthroplasty, comprising the steps of:
installing a rod or stem having a first end positioned within the intramedullary canal and a second end that remains exposed;
coupling a referencing fixture to the second end of the rod or stem;
measuring the extent of the lateral femoral condyle or trochlear region using the referencing fixture; and
resecting the distal femur in accordance with the extent of the lateral femoral condyle or trochlear region, as measured with the referencing fixture.

6. The method of claim 5, including the step of placing a spacer between the referencing fixture and the lateral femoral condyle or trochlear region.

7. When installing a prosthesis onto a distal femur having a trochlear region, an improvement to promote restoration with respect to the patellar femoral joint in the distal plane, comprising:
increasing the depth of the trochlea with increasing implant size so as to optimize the patella femoral mechanics.

8. A method of resecting a distal femur having prominent and non-prominent condyles separated by a trochlear region, the method comprising the steps of:
a) installing a fixture onto the distal femur which references the non-prominent condyle or trochlear region; and
b) resecting the femur in accordance with the reference made in (a).

9. The method of claim 8, wherein the step of installing a fixture onto the distal femur includes the step of:
placing an intramedullary rod in the distal femur having a movable guide; and
moving the guide until the guide touches the non-prominent condyle or trochlear region.

10. The method of claim 8, wherein:
the femur has a longitudinal axis; and
the resection is substantially transverse to the longitudinal axis.

11. The method of claim 8, wherein the resection is a distal cut.

12. Apparatus for resecting a distal femur having prominent and non-prominent condyles separated by a trochlear region, comprising:
a fixture including a movable member which references one of the non-prominent and trochlear regions; and
a cutting guide to resect the femur in accordance with the reference made using the fixture.

13. The apparatus of claim 12, wherein the fixture further includes:
an intramedullary rod; and
a reference guide movable on the rod.

14. The apparatus of claim 13, wherein the fixture further includes a medial-to-lateral slide to which the reference guide attaches, enabling either condyle or the trochlear region to be used as a reference for subsequent resection.

15. The apparatus of claim 12, further including a prosthesis installable on the distal femur in accordance with the resection made with the cutting guide.
